(19) United States
(12) Patent Application Publication
(10) Pub. No.: US 2023/0220325 A1
(43) Pub. Date: Jul. 13, 2023

(54) COMPOSITIONS AND METHODS FOR NEGATIVE SELECTION OF NAIVE T AND B CELLS WITH A SINGLE ANTIBODY

(71) Applicant: BioMagnetic Solutions LLC, State College, PA (US)

(72) Inventors: Qiuyan CHEN, State College, PA (US); Paul A. LIBERTI, State College, PA (US)

(73) Assignee: BioMagnetic Solutions LLC, State College, PA (US)

(21) Appl. No.: 17/999,987

(22) PCT Filed: May 28, 2021
(86) PCT No.: PCT/US2021/034817

§ 371 Date: Nov. 28, 2022

Related U.S. Application Data
(60) Provisional application No. 63/031,184, filed on May 28, 2020.

(51) Int. Cl. C12M 1/00; B03C 1/01; G01N 33/544

(52) U.S. Cl. CPC C12M 47/04; B03C 1/01; G01N 33/544

(57) ABSTRACT

Compositions and methods for the isolation of naive, untouched target cells of interest are disclosed.

### CROSS-REFERENCE TO RELATED APPLICATION

This application claims priority to U.S. Provisional Application 63/031,184 filed May 28, 2020, the entire disclosure being incorporated by reference herein as though set forth in full.

### FIELD OF THE INVENTION

This invention relates to the fields of immunology and magnetic separation and isolation of untouched immune cells e.g., T, B and NK cells, from biological fluids. Specifically, compositions and methods are provided for efficient separation of undesirable cell types from biological samples, leaving the cell type of interest in an untouched, naïve state, which can then be isolated and employed in a variety of therapeutic applications.

### BACKGROUND OF THE INVENTION

Several publications and patent documents are cited throughout the specification in order to describe the state of the art to which this invention pertains. Each of these citations is incorporated herein by reference as though set forth in full.

Immunotherapy involving the processes of selection, priming, expansion and transfection of T lymphocytes (T cells) to create chimeric antigen receptor (CAR) T cells is one of the most significant medical advances of this generation. Having demonstrated cures for certain Leukemias and Lymphomas with FDA approved products [for large B-cell lymphoma (Kymriah-Novartis) and for non-Hodgkin lymphomas (Yescarta—Gilead)], many other applications of CAR T cells are being explored and many clinical trials are being conducted worldwide (Kansagra A J. et al., Clinical Utilization of Chimeric Antigen Receptor T Cells in B Cell Acute Lymphoblastic Leukemia: An Expert Opinion from the European Society for Blood and Marrow Transplantation and the American Society for Blood and Marrow Transplantation. Biol Blood Marrow Transplant. 25:e76-85, 2019; Seif M., et al. CAR T Cells Beyond Cancer: Hope for Immunomodulatory Therapy of Infectious Diseases. Front Immunol. 10:2711, 2019). Very significant efforts are being made to apply such therapies to solid tumors as illustrated in a recent review (Mohanty R., et al. CAR T cell therapy: A new era for cancer treatment. Oncol Rep. 42:2183-2195, 2019).

The manufacture process for CAR T cells (living drugs) starts with the collection of peripheral blood mononuclear cells (PBMC) by apheresis methods from the patient or a donor, in the case of allogeneic CAR T, where those cells can either be (i) directly stimulated to activate and expand so that sufficient numbers of cells can be processed in subsequent steps to make sufficient product, or (ii) subjected to the selection of T cells or subsets such as CD4⁺ and CD8⁺T cells. There is a mounting body of evidence that cell selection as a starting manufacture step provides a better product. Furthermore, there is compelling evidence that the starting ratio of CD4⁺ and CD8⁺ T cells is important providing more durable therapies (Turtle CJ., et al. CD19 CAR-T cells of defined CD4⁺:CD8⁺ composition in adult B cell ALL patients. J Clin Invest. 126:2123-38, 2016).

There are several methods of T cell selection that have been used for producing CAR T cells. Even though there are many good biological reasons to start with ‘untouched’ T cells, most process involve positive selection employing targeting mAbs with specificity to CD3 receptors on T cells or in the case of positive selection of CD4⁺ or CD8⁺ T cells the appropriate mAb. Those mAbs can either be used in concert with some common capture instrument or be conjugated to a separation vehicle such as magnetic nanoparticles, micro bubbles with flotation capabilities, other solid support, or the mAbs can be fluorescently labeled so that labeled cells can be selected by flow cytometry methods. None of those methods produce untouched T cells. Further, when particles are employed in separation methods, removal is required before starting subsequent manufacture steps. If the particles are not removed, it must be demonstrated unequivocally that no adverse effects are caused by the presence of such particles on starting cells—a tedious and expensive task.

Employing positive selection methods for T cell and T cell subsets has certain advantages. For example, because most negative selection protocols require removing all non-T cell species from PBMC, available negative separation systems typically employ monoclonal antibody (mAb) cocktails containing as many as nine mAbs (minimally seven). These antibodies bind specific surface receptors for removal of non-target cells, e.g., CD14-monocytes, CD15-granulocytes, CD16-NK cells and granulocytes, CD19-B cell, CD34-stem cells, CD36-monocytes/macrophages/platelets, CD56-NK cells, CD123-cells of myeloid lineage and some B cells, (when T cells are to be negatively selected) and CD235a-RBCs.

Bearing in mind that, according to FDA guidelines, any entity, in this case mAbs, that contact precursor cells for therapeutic applications must be of the same quality as therapeutic mAbs for use in the human body. Accordingly, the startup costs for creating negative selection systems employing numerous mAbs for use in vivo present a cost burden on the healthcare system and on patients in need of such therapeutic products. The costs associated with performing such selections by cobbling together commercially available positive selection kits to remove unwanted cells totals about $20,000 for each Leukopak treated. Adding those costs to the current CAR T production costs is unacceptable.

### SUMMARY OF THE INVENTION

In accordance with the present invention a method of isolating an Fc receptor negative target cell fraction from a peripheral blood mononuclear cell (PBMC) preparation is disclosed. In one embodiment, a PBMC preparation rendered substantially free of endogenous or added IgG is provided. A single immunologically active capture agent which simultaneously binds to both Fc receptor-bearing cells and to epitopes on B cells is introduced where the capture agent is operably linked to a ferrofluid comprising magnetically responsive particles, and forms a magnetic cluster of Fc receptor bearing cells including B cells, monocytes, granulocytes, and platelets; said magnetic cluster of Fc receptor-bearing cells and B cells is then isolated from the preparation in a magnetic separator and the target cell fraction recovered in an essentially naïve untouched, condition.

In another embodiment for isolating Fc-receptor negative target cells in an untouched condition, a single immunologically active capture agent which simultaneously binds to both Fc receptor-bearing cells and to epitopes on B cells is introduced into a PBMC preparation, where the capture agent is operably linked to a first member of a specific binding pair. The preparation is then contacted with a ferrofluid comprising magnetically responsive particles operably linked to a second binding member, under conditions where a specific binding pair forms between said first and second binding pair members thereby forming a magnetic cluster of Fc receptor bearing cells selected from B cells, monocytes, granulocytes, and platelets. The magnetic cluster of cells is then isolated from the preparation in a magnetic separator, and the target cell fraction retrieved in an essentially naïve condition. B cell epitopes for use in the methods disclosed herein include, without limitation, CD19, CD20, IgG and CD32.

In yet another embodiment of a method for isolating Fc-receptor negative target cells in an untouched condition, an anti-human IgG and a capture agent comprising a first member of a specific binding pair, which is an Fab or F(ab)′_{μ}cells, each of said IgG and Fab or F(ab)′₂ having affinity for a B cell epitope, are introduced into a PBMC preparation having reduced levels of endogenous IgG. The PBMC preparation is contacted with a ferrofluid comprising magnetically responsive particles operably linked to a second binding pair member, under conditions where a specific binding pair forms between said first and second binding pair members, thereby forming a magnetic cluster of Fc receptor bearing cells including IgG-bound B cells, monocytes, granulocytes, and platelets. The magnetic cluster of cells is then isolated from said preparation in a magnetic separator, and the target cell fraction recovered in an essentially naïve condition.

In certain preferred embodiments, the target cells are CD3⁺ T cells. In other embodiments, where the specificity of the antibodies are changed, the clustering features described above can be used to isolate B cells. By providing the appropriate binding pair members, CD34⁺ stem cells, CD4⁺, CD8⁺, and NK cells can be isolated.

The capture agent for use in the methods described above includes for example, a monoclonal IgG antibody of mouse or human origin which comprises Fc regions which are bound by human FcγR, the antibody having binding affinity for an epitope on B cells, on non-target cells in cases where untouched T cells are to be recovered. In certain preferred embodiments, the antibody is IgG₁. In other approaches, IgG is added along with an immunologically active antibody fragment, (e.g., Fab), where the fragment is operably linked to a first member of a specific binding pair. In embodiments where FcγR bearing non-target cells are to be removed, such cells include monocytes, granulocytes, macrophages, dendritic cells, and NK cells.

While the use of biotin and streptavidin are exemplified herein, other useful binding pair members include without limitation, receptor-ligand, agonist-antagonist, lectin-carbohydrate, avidin-biotin, biotin analog-avidin, desthiobiotin-streptavidin, desthiobiotin-avidin, iminobiotin-streptavidin, and iminobiotin-avidin.

Also provided are methods for isolation of naïve CD4⁺ or CD8⁺ T cells from a peripheral blood mononuclear cell (PBMC) preparation. In an embodiment where naïve CD4⁺ cells are to isolated, a first immunologically active capture agent which simultaneously binds to both Fc receptor-bearing cells and to epitopes on B cells and a second immunologically active capture agent which binds to CD8⁺ T cells are introduced into the PBMC preparation. In this case, each of said first and second immunologically active capture agents are operably linked to magnetically responsive particles present in ferrofluid, which form a magnetic cluster of CD8⁺ T cells, B cells and said Fc receptor bearing cells. The magnetic cluster of cells is isolated from said preparation in a magnetic separator, and CD4⁺ T cells are recovered in an essentially naïve condition. In a situation where it is desirable to isolate CD8⁺ T cells, an anti-CD8 antibody is replaced with an anti-CD4 antibody.

The invention also provides a method for isolating naïve NK cells from a peripheral blood mononuclear cell (PBMC) preparation under conditions suitable for high affinity Fc-receptor binding. In this embodiment, a first immunologically active capture agent which simultaneously binds to both Fc receptor-bearing cells and to epitopes on B cells and a second immunologically active capture agent which binds to CD3⁺ T cells under conditions which promote high affinity FcR binding, each of said first and second immunologically active capture agents being operably linked to magnetically responsive particles present in ferrofluid and forming magnetic clusters of Fc-receptor bearing cells, B cells and CD3⁺ cells. The magnetic cluster is then isolated from the preparation in a magnetic separator, and naïve NK cells are recovered in an essentially naïve condition.

In yet another aspect, a method for isolating naïve CD34⁺ stem cells from a peripheral blood mononuclear cell (PBMC) preparation is provided. An exemplary method entails introducing into a PBMC preparation, an immunologically active capture agent which simultaneously binds to both Fc receptor-bearing cells and to epitopes on T cells, said immunologically active capture agent being operably linked to magnetically responsive particles present in ferrofluid; said Fc receptor-bearing cells, and T cells forming a magnetic cluster which is isolated from said preparation in a magnetic separator, thereby allowing for recovery of CD34³⁰ stem cells in an essentially naïve, untouched condition. In a preferred embodiment of this method, PBMC are isolated from a donor treated with G-CSF to cause hematopoietic stem cells to migrate from the bone marrow into peripheral blood.

### BRIEF DESCRIPTION OF THE DRAWINGS

FIG. 1 is a schematic diagram of the binding partners for negative selection of naïve, untouched CD3 cells described in the Examples below. Anti-CD32 mAb binds to CD32 expressing cells through the Fab paratope-CD32 epitope interaction and binds to other FcγRs through Fc-FcγR interactions. CD19 and CD20 are B cell specific receptors, anti-CD19 and CD20 bind B cells through specific CD19 and CD20 epitope, respectively, and also bind to other cells through Fc-FcγR interaction. sIg is also a B cell surface specific receptor, anti-IgG mAb bind B cells through the sIg epitopes and binds to other cells through Fc-FcγR interactions. Moreover, anti-IgG can also be used for binding to plasma free Ig which labels FcγR expressing cells. When anti-IgG antibody is used, the antibody can be in a full-length IgG format or in an Fab/F(ab′) format having affinity for plasma free Ig to label the FcγRC.

FIG. 2 shows histograms obtained after cells were labeled and separated with MAH-Ig-FF and subjected to flow cytometry analysis. Cells were gated on CD45-FITC and measure the CD3, CD11and CD19 positive cells after cell selection.

### DETAILED DESCRIPTION OF THE INVENTION

Increasing clinical data demonstrates the success of the adoptive transfer of genetically engineered T cells, such as chimeric antigen receptor (CAR) T cell mediated immune therapy, to effectively treat cancer and autoimmune diseases. To date, manufacture of immune cells mainly employs positive selection of T cells that is based on mAb labeling of the specific receptor expressed on those cells. This strategy can result in altered expression of different genes because the positive selection process requires contacting cells with reagents which can induce unwanted or premature cell activation and mediate activation-induced cell death following isolation. Furthermore, there is anecdotal information that positive cell selection can lead to antibody dependent cellular cytotoxicity shortly after transfer into the recipient. It is quite possible that these shortcomings adversely affect the potency and long-term persistence of adopted immune cells. Thus, an alternative strategy, employing simplified negative selection of desired cell types, e.g., naïve T, B, NK or CD34⁺ stem cells, would be of great value and reduce undesirable activation of target cells.

The composition of leukocytes in PBMC/Leukopaks is: T cells (45-60%), B cells (5-15%), monocytes (10-30%), granulocytes (0.6-10%) and NK cells (5-10%). To date there are no negative selection protocols for preparing CAR T cells in clinical applications. However, there are several conventional research level negative selection options for PBMC/Leukopaks using magnetic separation. For example, in the Miltenyi Biotech (Bergisch Gladbach, Germany) pan-T cell selection kit (#130-096-535), the antibody cocktail contains nine antibodies (CD14, CD15, CD16, CD19, CD34, CD36, CD56, CD123, and CD235a) in order to remove non-T cells from PBMC; similarly in a Dynal bead-based CD3 negative selection product, seven antibodies are involved (CD14, CD16, CD19, CD36, CD56, CD123 and CD235a), which gives comparable purity (˜95% and up) (Dynabeads™ Untouched™ Human T Cells Kit, #11344D). One report that uses three antibodies, CD14, CD19 and CD56 to negatively enrich CD3⁺ T cells gives similar results, however, Ficoll purification is required before separation to remove granulocytes (Janssen W., et al. A simple large scale method for T cell enrichment by negative selection in preparation for viral transduction. Cytotherapy 19:S38, 2017).

In previous studies on CD3 positive T cell selection (Liberti P A, Riter D W, Khristov T R. A novel low cost high yield clinical scale cell separator. Cell Gene Therapy Insight. 4:581-600, 2018), an indirect method of magnetically labeling T cells was employed to effect positive immunomagnetic separation. In those studies, PBMC were first incubated with anti-CD3 mAb and subsequently magnetically labeled with common capture versions of our 135-165 nm proprietary highly magnetic nanoparticles (alternatively referred to as ferrofluid or FF) (Liberti et al., U.S. Pat. Nos. 5,698,271; 6,120,856). Preliminary studies were initiated using a common capture agent, a rat anti-mouse Fc (RAM) mAb coupled to FF. That system consistently resulted in a T cell product of excellent yields of CD3⁺ cells (>75%) but that were repeatedly contaminated with different levels of monocytes (10-95% of monocytes in the PBMC preparation). On the presumption that RAM-FF was interacting with FcRs, significant attempts were made to inhibit monocyte binding which included pre-incubation of PBMC with heat aggregated human IgG (HAIG) as well as FcR blocking antibodies. Regardless of the level of HAIG, monocyte binding could not be inhibited whereas Fc blocking antibodies were effective. However, the cost of employing such blocker made it prohibitive to create a commercially reasonably priced separation kit.

Analysis of the many different RAM-FF preparations used in those early positive selection T cell studies suggested a strong correlation between the levels of RAM used for conjugation of FF and monocyte contamination (capture). The analysis indicated that at some level of RAM conjugation to FF, these nanoparticles bind FcγR bearing cells (FcγRC) avidly indicating an important role for the proximity of Fc portions of RAM in its coupling to FF and other solid surfaces or supports. The role of RAM Fc proximity on solid supports such as our FF was experimentally demonstrated in our laboratories by effectively saturating the surface of FF with RAM (4000-7000 IgG/particle) and comparing its affinity for monocytes to that of low levels of conjugation (500-1000 IgG/particle) as well as other conjugated non-immunological proteins. FF prepared with high conjugated levels of RAM was capable of completing depleting monocytes from aliquots of apheresis product. Furthermore, such depletions can be undertaken with the same concentration of FF that would be used in mAb targeted cell separations. Note RAM is of IgG₁ subtype. Additionally, when PBMC were incubated with FF conjugated with bovine or human serum albumin (BSA, HSA), no monocytes could magnetically be removed regardless of the level of conjugation.

To determine if monocytes were ingesting RAM-FF rather than RAIVIFc:FcγR binding interactions to create magnetically responsive cells, experiments were undertaken at 0° C. and produced the same results as those completed at room temperature. When experiments were undertaken with lower levels of RAM conjugated to FF, lower levels of monocytes were retrieved. All of these findings indicate that RAM-FF:monocyte interactions are largely of a binding nature and either requires or is augmented by the close proximity of RAM Fc regions.

The foregoing discoveries prompted us to investigate the following hypothesis: at sufficiently high conjugation levels of a single antibody to a solid support, it should be possible to perform an efficient CD3⁺ T cell negative selection with a single antibody of IgG sub-class, because at such levels, Fc portions of such antibodies would be in sufficiently close proximity to act as avid binders of FcγRs and at the same time the specificity of that antibody could be used to bind at least one other cell specificity, viz., B cells. Thus, proximity of Fc on solid supports enhances avidity via multivalent attachments to near-neighbor FcγR. To further investigate, T cell negative selection with a single mAb, a mouse anti-human IgG antibody of IgG₁ isotype was conjugated to FF at high levels (4000-7000 mAb/particle). The working hypothesis for this experiment was that the Fab variable domain would bind to B cells because B cells express surface IgG, and the Fc region of this antibody in close proximity on a surface would bind to the FcγR expressed on monocytes, macrophages, dendritic cells (DCs), granulocytes, natural killer (NK) cells, and also B cells as B cell expression FCγRIM

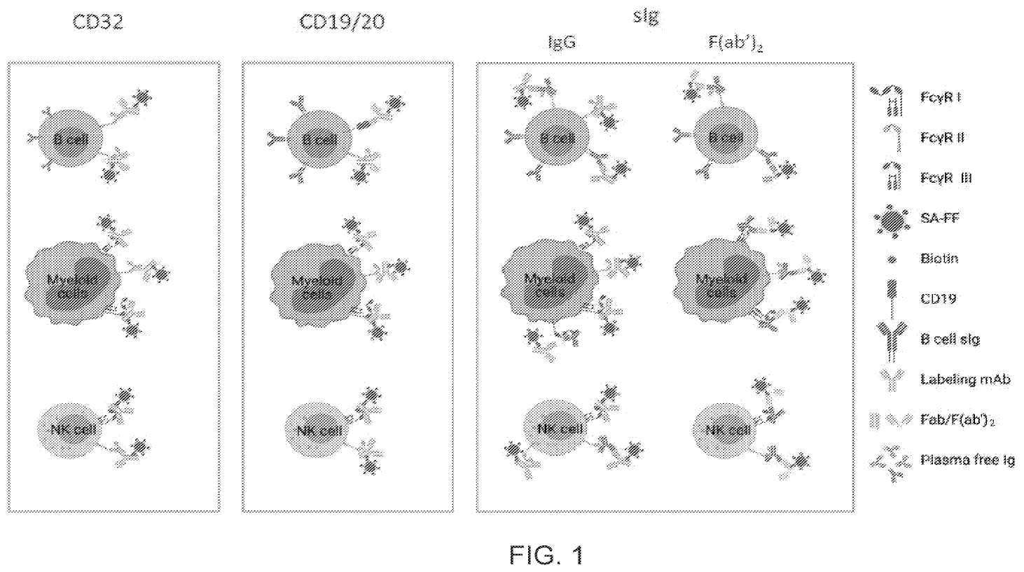

Experiments performed with many permutations and controls proved the hypothesis and demonstrated that with this strategy, we could repeatedly negatively isolate naïve CD3⁺ T cells with purity of 95% and above. For certain experiments, PBMC preparations were substantially free of endogenous IgG as they were washed by three cycles of centrifugations to avoid endogenous IgG clearly would react with the anti-human IgG antibody. When human IgG was added into such PBMC preparations at levels greater than about 4 to 10 μg/mL, T cell recovery decreased while B cell contamination increased. Given that an anti-human IgG was used to label B cells in those experiments we reasoned that that endogenous IgG would effectively neutralize the cell labeling capability of anti-human IgG antibody. In embodiments, when Fab or Fab like fragments linked by disulfides are used, e.g., F(ab′)₂, a low levels of endogenous IgG would facilitate selection, i.e.,the biotinylated Fab or F(ab′)₂ would bind to B cell surface IgG to label B cells, and Fab/F(ab′)₂ could also bind to free plasma IgG forming F(ab′)₂-IgG complex and labeling the FcγRC for removal (FIG. 1). Rendering a sample “substantially IgG free” entails at least three washes and resuspensions. When Fab/F(ab′)₂ are used, cells are only washed 1×x or 2×, thereby retaining residual plasma IgG in the sample.

Since anti-human IgG antibody can bind to endogenous IgG in the cell product as well as Ig bound to FcγRC, to simplify the model, an anti-CD19 mAb was coupled to FF at high levels of conjugation (4000-7000 mAb/particle) and used for negative selections of PBMC that was not devoid of endogenous IgG. This experiment also led to the production of naïve T cells in high purity (>92%). From this conceptually simple experiment compared with the anti-human Ig experiment above, we surmised that one component that specifically reacts with B cells and another with FcγRC via what is most likely aggregated Fc, is sufficient to produce naïve T cells.

Using a single antibody to enrich a target cell significantly simplifies the cell labeling procedure, dramatically reduces the cost of involving many antibodies as in conventional protocols and results in an incredibly rapid process. The attractiveness of this concept, supported by experimental data disclosed below, will greatly accelerate translation into clinically relevant products. The same strategy can be also used to simplify the negative selection of CD4⁺ or CD8⁺ T cells, B cells and NK cells which can be accomplished by the addition of one other mAb to the system as described below.

As described above, our initial positive T cell selections from PBMC were undertaken by indirect magnetic labeling methods using steps of (i) anti-CD3 mAb incubation, (ii) removal of unbound mAb, and (iii) incubation with RAM-FF. The monocyte contamination in these selection protocols was completely unacceptable. If instead T cells were labeled with biotinylated-anti-CD3 mAb, followed by unbound mAb removal, SA-FF incubation and magnetic separation, for similar positive selection experiments, dramatic improvement in monocyte contamination was observed but still present at unacceptable levels. This issue was resolved by first incubating PBMC, which were substantially free of endogenous IgG, with human IgG at just 1.0 mg/mL, followed by incubation with biotin-anti CD3 antibody, removal of unbound mAb and subsequent magnetic labeling with SA-FF. Those experiments resulted in T cell preparations essentially free of monocyte contamination. These results suggest that interactions of biotinylated-anti-CD3 mAb with FcR, particularly high affinity receptors, might be playing a role in the monocyte contamination in the absence of added IgG. It is important to keep in mind that it took 1 mg/mL of human IgG to prevent monocyte contamination in the above described experiment, i.e., about 1000 to 1 that of the labeling mAb.

We then considered the possibility that biotin-anti-CD3 mAb incubated with PBMC in the absence of other Igs might either be exchanging with endogenous IgG bound to FcγR or that there are sufficient numbers of ‘empty’ FcR such that added labeling mAb could occupy such sites leading to targetable anchors for labeling FcγRC. Since K_{d} of different FcγR binding reactions to monomeric IgG range from 10⁻⁶ to 10⁻⁹ M, IgGs should be bound with sufficient energy to serve as a labeling agent. Furthermore, because of the multivalence of our SA-FF the opportunity to create enhanced binding energy of FcγRC and SA-FF via multivalent attachment to biotinylated mAb on FcγRC, makes the possibility of mAb's Fc—FcγR interactions a contributing factor.

These findings are particularly relevant to any negative separation experiment. The preferred protocol is retaining unbound labeling mAb in the reaction mixture prior to the addition of common capture agent. That gives such agents, in this case SA-FF, opportunity for reactions with mAb labeled B cell as well as any other component bearing biotin. For example, in the case of using a biotin-anti-CD19 mAb, incubation of mAb with PBMC could lead to mAb labeled B cells and mAb labeling of FcγR on FcγRC. Upon addition of a common capture agent like SA-FF several reactions could occur, including the latter binding to mAb labeled B cells, binding to any FcγRC bearing biotin as well as binding to unbound biotin-anti-CD19. Therefore, during the magnetic labeling incubation period, in addition to labeling reactions with B cells there is also the formation of a new species, viz., complexes of SA-FF with bound antibodies. Based on the RAM-FF data, described above, we predicted that this complex would avidly bind to FcγRC. Thus, employing mAbs specific for B cells (such as CD19, CD20 or IgG), in indirect selection protocols likely involves a host of complex reactions where common capture agents can play several advantageous roles. For example, the SA multivalence on a nanoparticle, or surface, could promote labeling FcRC via biotin-antibody bound to that FcRC in which case multivalence of SA-FF enhances binding strength to the point where such FcRC can be captured. That same multivalence of SA-FF in concert with its reaction with unbound biotin-mAb creates species that can react with unfilled FcR, thus creating another mechanism for labeling FcR cells (FcRC) for subsequent removal.

Based on our positive selection T cell studies with SA-FF and the effect of added human IgG on diminishing monocyte contamination, it would seem likely that added IgG could negate multivalent SA-FF binding to biotinylated mAb bound to FcγR as at high enough concentrations of added IgG those mAbs would be expected to be competed off FcγR especially after removal of unbound biotin-anti-CD3 mAb in those experiments.

To gain some insights on the possible mechanisms involved in FcγRC removal, the following negative T cell selection experiment was undertaken: biotinylated anti-CD19 mAb was incubated with PBMC in the absence of IgG followed by incubation with SA-FF and magnetic separation. T cell purities of >92% were obtained with little monocyte contamination of the T cell fraction. Presence of IgG (0.125 -1 mg/mL) did not significantly change monocyte depletion effect, as anticipated. Moreover, with increased amount of biotinylated anti-CD19 mAb the removal of monocytes became more complete (e.g., 2-4 μg/mL is better than <2 μg mL), and the CD3 purity was higher. Based on these results, it appears that biotin-mAb, complexed with surfaces capable of binding them in a closed packed manner, are able to avidly bind FcRC.

These disclosures demonstrate that there are various ways to engage FcγR in cell separations in a very positive manner, creating several routes to isolation of naïve, untouched cells via simple, reagent sparing manner.

### Definitions:

In order that the present disclosure may be more readily understood, certain terms are first defined. As used in this application, except as otherwise expressly provided herein, each of the following terms shall have the meaning set forth below. Additional definitions are set forth throughout the application.

The term “biological sample” includes, without limitation, cell-containing bodily, fluids, peripheral blood, tissue homogenates, aspirates, and any other source of rare cells that is obtainable from a human subject.

The term “determinant”, when used in reference to any of the target cells described herein, may be specifically bound by a biospecific ligand or a biospecific reagent, and refers to that portion of the target cell involved in, and responsible for, selective binding to a specific binding substance, the presence of which is required for selective binding to occur. In fundamental terms, determinants are molecular contact regions on target cells that are recognized by receptors in specific binding pair reactions.

The term “specific binding pair” as used herein includes antigen-antibody, receptor-hormone, receptor-ligand, agonist-antagonist, lectin-carbohydrate, nucleic acid (RNA or DNA) hybridizing sequences, Fc receptor or mouse IgG-protein A, avidin-biotin, streptavidin-biotin and virus-receptor interactions. Various other determinant-specific binding substance combinations are contemplated for use in practicing the methods of this invention, such as will be apparent to those skilled in the art. When a first member of a specific binding pair such as an anti-CD3 mAb binds to its second member, CD3 epitopes on T cells, such reactions are referred to as “labeling reactions” and those T cells are considered labeled by mAb.

“Positive selection” refers to purification from a mixture of different attachment of a first member of a specific binding pair that selectively binds to the second member of a second binding pair present on the target cell type of interest, thereby allowing the cell to isolated from the mixture. A variety of means and methods for performing positive selections, i.e., purifying the entity of interest, employing the second member of a specific binding pair are well known in the art.

“Negative selection” refers to purification of a target cell type from a mixture of different cell types by attachment of one or more first members of one or more specific binding pairs to each and every cell type in the mixture with the exception of the cell type of interest. Specific binding pair reactions employing the second member of a binding pair allow those entities bearing the first member of a binding pair to be separated from the mixture, leaving behind the entity of interest. Means and methods for performing such separations are well known in the art. The portion of the mixture that is left behind is referred to as the negative fraction. “Essentially naïve or untouched condition” refers to that subset of cells that have not been contacted by any specific binding pair member.

“T cells” shall mean CD3⁺ cells. By definition, a negative selection of CD3⁺ cells produce naïve T cells that have not been contacted with any member of a specific binding pair.

An “Fc-receptor negative target cell” is a target cells that expresses little or no Fc receptor.

In positive and negative selections, cell types that are to be retrieved or eliminated, respectively, are typically contacted with one member of a specific binding pair such as an epitope on those cells reacting with corresponding antibodies or some agent that specifically binds to that epitope with high affinity, the second member of the specific binding pair. Such binding pair reactions of high affinity are often referred to as “labeling reactions”.

The term “antibody” includes, without limitation, a glycoprotein immunoglobulin which binds specifically to an antigen. In general, an antibody can comprise at least two heavy (H) chains and two light (L) chains interconnected by disulfide bonds, or an antigen binding molecule thereof. Each H chain comprises a heavy chain variable region and a heavy chain constant region. The heavy chain constant region comprises three constant domains, CH1, CH2 and CH3. Each light chain comprises a light chain variable region and a light chain constant region. The variable regions of the heavy and light chains contain a binding domain that interacts with an antigen. The portions of antibody molecules formed by the interaction of light and heavy chain variable regions (VL and VH) as well as interactions of their constant regions (CL and CH1) is referred to as Fab or Fab fragments (fraction antigen binding). Papain digestion of antibodies leads to the production of Fab fragments (2 per molecule) and a fraction that crystalizes known as Fc (fraction crystalized). Fc fragments consist of heavy chain domains below the hinge region and are formed by the interaction of the CH2 domains with each other and similarly the CH3 domains. Pepsin digestion of antibodies cleaves antibody molecules below the hinge region, leaving the disulfide bonds linking heavy chains intact. Thus, two Fab like fragments linked by disulfides are produced and referred to as (Fab′)₂. When those disulfides are reduced single antigen binding fragments are produced (Fab′). The hinge region and CH2 domain (a region just below and proximal to the hinge region) of heavy chains contains the regions of the antibody molecule that binds to Fc Receptors (FcR) on cells. Because pepsin digestion of antibodies does not always result in uniform cleavage, some (Fab′)₂ preparations can contain the FcR binding sequences.

Antibodies or immunoglobulins (Ig) may be derived from any of the commonly known isotypes, including but not limited to IgG, IgM, IgE, IgA and secretory IgA. IgG sub-classes are also well known to those in the art and include but are not limited to human IgG₁, IgG₂, IgG₃ and IgG₄, or mouse IgG₁, IgG₂ and IgG₃. The term “antibody” includes, by way of example, both naturally occurring and non-naturally occurring antibodies; monoclonal and polyclonal antibodies; chimeric and humanized antibodies; human or non-human antibodies; wholly synthetic antibodies; recombinantly produced antibodies; immunoglobulins, an antibody light chain monomer, an antibody heavy chain monomer, an antibody light chain dimer, an antibody heavy chain dimer, an antibody light chain-heavy chain pair, intrabodies, antibody fusions, heteroconjugate antibodies, single domain antibodies, monovalent antibodies, single chain antibodies or single-chain Fvs (scFv), affibodies, Fab fragments, F(ab′)₂ fragments, disulfide-linked Fvs (sdFv), minibodies, domain antibodies, synthetic antibodies (sometimes referred to herein as “antibody mimetics”), and antigen-binding fragments of any of the above. Antibodies as defined above can be obtained from any species.

Cells which express the Fc receptor (FcRC) are distinguished from other hematopoietic cells by their ability to adhere to antibody-antigen complexes. FcRs bind to amino acid sequences located mainly in the CH2 domain of certain subclasses of antibodies. FcR bind to sequences on antibody heavy chains that are in the lower part of the hinge or below, and in proximity to the hinge region of CH2 of certain subclasses of antibodies. FcγR is a class of FcR that specifically bind to only IgG antibodies of certain subclasses. These trans-membrane molecules recognize the Fc region of several immunoglobulin (Ig) classes and sub-classes. The terminology for FcR for different isotypes and their corresponding CD designation is as follows: for IgG (FcγRI/CD64, FcγRII/CD32, and FcγRIII/CD16), IgE (FcεRI), IgA (FcαRI/CD89), IgM (FcμR), and IgA/IgM (Fcα/μR). The phrase “substantially free of FcRs” refers to a cell that will not adhere to a surface bearing immune complexes where those complexes are formed from Ig isotypes and sub-classes thereof that are known to bind to FcR.

“Isotype” refers to the antibody class or subclass (e.g., IgM or IgG₁) that is encoded by the heavy chain constant region genes.

The term “detectably label” refers to any substance whose detection or measurement, either directly or indirectly, by physical or chemical means, is indicative of the presence of the target cell in the test sample. Representative examples of useful detectable labels, include, but are not limited to the following: molecules or ions directly or indirectly detectable based on light absorbance, fluorescence, reflectance, light scatter, phosphorescence, or luminescence properties; molecules or ions detectable by their radioactive properties; molecules or ions detectable by their nuclear magnetic resonance or paramagnetic properties. Included among the group of molecules indirectly detectable based on light absorbance or fluorescence, for example, are various enzymes which cause appropriate substrates to convert, e.g., from non-light absorbing to light absorbing molecules, or from non-fluorescent to fluorescent molecules.

The phrase “to the substantial exclusion of” refers to the specificity of the binding reaction between the biospecific ligand (e.g., mAb) or biospecific reagent (e.g., biotin and streptavidin) and its corresponding target determinant (e.g., cellular receptor on cell of interest). Biospecific ligands and reagents have specific binding activity for their target determinant yet may also exhibit a low level of non-specific binding to other sample components.

The term “enrichment” as used herein refers to the enrichment of target T and B cells from a biological sample.

The preferred magnetic particles for use in carrying out this invention are particles that behave as colloids. Such particles are characterized by their sub-micron particle size, which is generally less than about 200 nanometers (nm) (0.20 microns), and their stability to gravitational separation from solution for extended periods of time. In addition to the many other advantages, this size range makes them essentially invisible to analytical techniques commonly applied to cell analysis. Particles within the range of 90-150 nm and having between 70-90% magnetic mass are contemplated for use in the present invention. Suitable magnetic particles are composed of a crystalline core of superparamagnetic material surrounded by molecules which are bonded, e.g., physically absorbed or covalently attached, to the magnetic core and which confer stabilizing colloidal properties. The coating material should preferably be applied in an amount effective to prevent non-specific interactions between biological macromolecules found in the sample and the magnetic cores. Such biological macromolecules may include sialic acid residues on the surface of non-target cells, lectins, glyproteins and other membrane components. In addition, the material should contain as much magnetic mass/nanoparticle as possible. The size of the magnetic crystals comprising the core is sufficiently small that they do not contain a complete magnetic domain. The size of the nanoparticles is sufficiently small such that their Brownian energy exceeds their magnetic moment. As a consequence, North Pole, South Pole alignment and subsequent mutual attraction/repulsion of these colloidal magnetic particles does not appear to occur even in moderately strong magnetic fields, contributing to their solution stability. Finally, the magnetic particles should be separable in high magnetic gradient external field separators. That characteristic facilitates sample handling and provides economic advantages over the more complicated internal gradient columns loaded with ferromagnetic beads or steel wool. Magnetic particles having the above-described properties can be prepared by modification of base materials described in U.S. Pat. Nos. 4,795,698, 5,597,531 and 5,698,271. Their preparation from those base materials is described below.

### Efficient Isolation of Naïve, Untouched Target Cells

Described herein are methods of preparing naïve cells, e.g., T cells, for use in genetic engineering and therapeutic methods such as adoptive cell therapy. Specifically, in some embodiments, the methods use or generate compositions that contain a plurality of different cell populations or types of cells, such as isolated CD4⁺ and/or CD8⁺ T cell populations. In some embodiments, the methods include steps for isolating one or more cell populations. The cells that are subjected to the methods described herein generally are isolated from a sample derived from a mammalian subject, preferably a human subject.

We have discovered that there are multiple ways in which to utilize FcRs on FcRC to perform negative selections of several important cell populations with minimal numbers of specific antibodies. In the case of T cells, we have found that a single antibody, preferably of the IgG class, whose amino acid residues below and proximal to the hinge region, CH2 domain, or intact Fc can create binding reactions with FcγRCs. The Fab portions of such antibodies react with unique epitopes on B cells and can be used effectively to label all cells except naïve and memory T cells in PBMC preparations, in the substantial absence of endogenous or added IgG other than the labeling antibody and in other cases with IgG present. In concert with a spectrum of well-known cell separation technologies, e.g., specific binding pair reactions employing solid supports and flow cytometry type of cell sorting methods, our discovery enables the purification of naïve T cells with a single mAb or highly specific polyclonal antibodies. In other words, FcγRs expressed on monocytes, macrophages, dendritic cells (DCs), granulocytes and NK cells as well as Fc portions of antibodies that interact with FcγR can be used inventively and advantageously for cell separation and purification of naïve T cells. When that discovery is combined with the employment of a single mAb directed to B cell epitopes, such as surface Ig, CD19, CD20 or CD32 on B cells, a simple, efficient and incredibly economic methodology for preparing naïve T cells is achieved. In combination with a second mAb directed to CD4⁺ or CD8⁺ cells, naïve CD4⁺ or CD8⁺ cells are readily prepared. There are multifold embodiments of this invention.

In one embodiment for producing naïve T cells, the substantial labeling of all but naïve T cells in PBMC for subsequent removal, is achieved via the attachment of Fc fragments, from antibody classes that bind to FcγR, to a solid support plus one other entity that can bind to B cells with sufficient binding energy, in both cases, to remove FcγRC and B cells, respectively, from a suspension. That can be accomplished with a single antibody with unique B cell specificity and of a subclass that binds to FcγR. Notably, we have determined that the density of Fc on such surfaces affects binding interactions with FcγR or Ig bound to FcγR, and that proximity of Fc on that surface must be considered. We theorize that there is an enhancement of those interactions via multivalent binding interactions which are well known in biological processes.

There are several determinants or epitopes specific to B cells that can be targeted for the above purpose, such as CD19, CD20 and surface Igs. The expression of these B cell specific epitopes is quite stable during B cell development. Another suitable epitope is CD32, which is preferred among other B cell specific epitopes because of its broad expression. CD32 is expressed on B cells and other WBCs, excluding T cells. This broad expression offers an effective approach for labeling for all FcγRC via strong Fab/CD32 epitope binding and the Fc/FcγR binding. In the present application, we describe several mAbs which are suitable for this task. Simply coupling any of those FcγR reactive mAbs to surfaces in appropriate densities create solid supports that will bind B cells and FcγRC. In the case of coupling one of those mAbs to a petri dish or similar vessel, researchers or diagnosticians can readily prepare naïve T cells simply by panning out unwanted cells. In the case of coupling such mAbs to nanoparticles, such as FF employed here, other magnetic particles or nano/micro entities with flotation capabilities, simple means for larger scale separations are enabled. There are various ways in which mAbs can be attached onto surfaces directly or coupled via a suitable linker. They can be coupled via linkers which could be advantageous but as we show direct coupling works well.

It will be appreciated that fragments of antibodies could be employed in the above embodiment. Methods of producing Fab and Fc fragments have been known for nearly 50 years and are readily produced as are methods for preparing CH2 domain which is the main portion of the IgG molecule that binds to FcγR. Thus, either Fc or CH2 fragments or even amino acid sequences of the hinge region and just below and proximal to the hinge region (Kiyoshi M., et al. Structural basis for binding of human IgG1 to its high-affinity human receptor FcγRI. Nature Communications, (2015), 6:6866) could be immobilized along with an anti-B cell specific entity to produce strong and specific solid supports for capturing and subsequent removal of FcγRCs and B cells. Those fragments can be linked directly but it could be more efficacious to employ linkers as the latter approach would result in more stereo-chemical binding opportunities.

In another embodiment for producing naïve T cells that would have some of the advantages afforded by indirect labeling processes, as well as the merits of a common capture agent, PBMC would be incubated with mAb directed to a B cell epitope such as anti-CD19, -CD20, anti-human Ig, or anti-CD32. That would be followed by incubation with a common capture agent on some appropriate support bearing an anti-Fc to the species of origin of the labeling mAbs. The requirement for such an anti-Fc is that it must be of the subtype capable of strong interactions with FcγR which are well known in the art. Even though, for clinical use, this embodiment requires two mAbs (labeling mAb and common capture mAb), there is efficient use of the labeling mAb, i.e., using anti-B cell mAb in its molecular form, while the second mAb on the common capture agent can be used in that way in many other specific separation processes.

We have employed a monoclonal rat anti-mouse Fc (RAM) coupled at high density to FF for this purpose and, as already mentioned, we have used SA bound to a solid support in conjunction with biotinylated targeting mAbs as well.

It is noteworthy that in the case where RAM coupled to a solid support is used for labeling of FcγRC in the indirect protocol there will likely be two species that are involved in that labeling reaction, viz., the RAM-solid support and that component bound to targeting antibodies, providing they are of the appropriate subclass. Alternatively, when SA-conjugated solid supports, or other specific binding pair reactions, are used there is likely a different mechanism. In these cases, the common capture agent most likely becomes a labeling agent for FcγRC when they bind targeting mAbs and possibly also, via their multivalence, by binding to biotin labeled mAbs associated with FcγR. There are other specific binding pair reactions known in the art such as DNP/anti-DNP, fluorescein/anti-fluorescein, biotin/anti-biotin and arsanilic acid/anti-arsanilic acid that can be used in place of biotin/streptavidin, however, the strength of the biotin/SA binding pair is as close to that of a covalent bond as is readily available. In this kind of negative selection process, there is no need to reverse the labeling, if in the future there should be a need to reverse reactions with those cells that are removed, binding pairs having less binding avidity can be used, e.g., desthiobiotin and streptavidin or a biotin-anti-biotin binding pair reaction which can be dissociated with avidin or streptavidin. Other methods that break the bonds between mAb and biotin, or streptavidin and HSA can also be considered.

Because PBMC can be classified into three subgroups or fractions, viz., naïve T, B cells and FcγRC, any method that can remove the latter two groups should result in isolation of naïve T cells. The above embodiments exploit the commonality of FcγR on many cell types, and targeting such cells, in combination with anti-B cell mAbs coupled to solid supports would with appropriate processing produce naïve T cells. Additionally, the labeling and separation processing steps could be undertaken with the indirect procedure employing biotinylated mAbs or their binding fragments in concert with a SA common capture agent. SA could either be coupled to any of the nano/micro particles mentioned or could be linked to column packing materials such as Sepharose or fibers and others known in the art (See, e.g., Etchells and Peterson, U.S. Pat No. 5,215,926).

As mentioned above that PBMC can be classified into three subgroups or fractions, viz., naïve T, B cells and FcγR cells, and using one antibody specifically targeting B cell epitope is effective to isolate CD3⁺ T cells to a high purity. In fact, B cells also express low affinity FcγR, FcγRIM, therefore the PBMC also can be classified in two classes, e.g., T cells and FcγR cells. Using antibodies targeting FcγRs will also offer the ability to remove non-T cells with both of its Fab and Fc portion to bind FcγRs. It is known that CD32, also named as FcγRII, is widely expressed on B cells, monocytes, granulocytes, dendritic cells, NK cells and platelets, so an anti-human CD32 antibody of the IgG class should be able to remove all the FcγRII expressing cells using the Fab region and other FcγR bearing cells using Fc region and the appropriate conjugation level of FF, leaving only T cells in the fraction.

As previously mentioned, in the embodiments where a single antibody, i.e., a mAb with unique specificity to B cells, is employed to produce naïve T cells, if a second mAb that is directed to CD4⁺ or CD8⁺ T cells were added, it would be clear that either naïve CD4⁺ or CD8⁺ cells would be produced via an appropriate separation system. In another embodiment, it would similarly be possible to recover either naïve CD4⁺ or CD8⁺ cells along with one or the other of those cells labeled with mAb in one separation scheme. In other words, a method is provided for obtaining one of those naïve subsets untouched and the other labeled with mAb which can be undertaken in a single magnetic or buoyancy separation. For example, if, in one case, a mAb cocktail of biotinylated anti-B cell and desthiobiotin anti-CD4 is mixed with PBMC, all FcRC, B cells and CD4⁺ cells would be magnetically labeled upon addition and incubation with SA solid support. After magnetic separation, CD8⁺ cells would remain in the supernatant or fluid phase, devoid of FcRC, B cells and CD4⁺ cells. CD8⁺ in suspension are thus easily recovered. Magnetically separated cells would, in this case, contain CD4⁺ cells ‘positively selected’. Those cells could readily be released by the addition of biotin that would break the desthiobiotin: SA bonds. Were the separation undertaken on a device capable of magnetically collecting cells over a large area and cells that are quite uniformly layered as is the case with a system we have developed (WO 2016/183032 Al), good recoveries of those CD4⁺ cells would be expected. Recovery of naïve CD4⁺ cells by this method, if desired, can be accomplished by using a desthiobiotin anti-CD8 mAb instead of the anti-CD4 mAb. In addition to desthiobiotin dissociation means via biotin displacement, methods for dissociating biotin/anti-biotin reactions are well known in the art (Lund, G. and Wegmann, T., U.S. Pat. No. 5,518,882; Brieden, J. and Dose, C. US Pat No. 20140113315A1). Hence, there are several ways in which to recover mAb labeled CD4⁺ or CD8⁺ cells in the above scheme.

In addition to all of the above embodiments disclosed herein, there is another variable that could be employed that extends these concepts and methods even further, making it possible to isolate naïve NK cells and even naïve B cells. As mentioned above, since leukocytes with the exception of naïve T cells express FcγRs and because there is a very substantial difference in the dissociation constants for high and low affinity receptors (Mkaddem S., et al. “Understanding Fc receptor involvement in inflammatory diseases”: From mechanisms to new therapeutic tools. Front Immunol. 10:811, 2019; Chauhan A.K., Human CD4⁺ T-cells: A role for low-affinity Fc receptors. Front Immunol. 7:215, 2016), optimized negative separation conditions can be achieved by altering the concentrations of mAbs used for labeling PBMC preparations. We have hypothesized that high affinity FcγR can be selectively labeled by interaction with Fc on a solid support that are clustered to lesser extent than those that might be required for sufficiently strong binding to lower affinity FcγR. Thus, the isolation of FcγRC bearing only low affinity FcγR is now possible.

Another consequence of the complexes formed when appropriate mAbs interact with multivalent binding surfaces such as SA-FF and their subsequent interaction with FcR is that platelets also bear FcγR. Thus, when PBMC is incubated with, for example, a biotinylated mAb of appropriate isotype at sufficiently high concentration (>2 μg/mL, e.g., 2, 2.5, 3, 3.5 4, 4.5, 5 μ/ml) followed by SA-FF, platelets also become biotin labeled. Therefore, separation with a common capture magnetic nanoparticle, as above, or some other means that employs a solid support, or other separation substrate, will also remove platelets from the negative fraction. Since platelet contamination can be an issue even in preparing PBMC devoid of most endogenous IgG, they can be removed via their FcγR. Hence, the present invention not only affords means for creating methods for preparing naïve T cells and other cells, but also saves time and money as the need to do any significant processing of blood products before selection is initiated is eliminated.

In addition to all of the foregoing, the principle that FcγRC can be readily depleted as disclosed herein, enables other important clinical strategies. For example, currently, CD34⁺ stem cells are isolated via positive selection with anti-CD34 mAbs. It would be desirable to be able to process such cells for transplants or for genetic engineered therapies starting with untouched or naïve cells. Using current practices, a negative selection could be accomplished but that would take 7-9 mAbs to remove all but CD34⁺ cells. From an economic viewpoint that is not a viable approach. On the other hand, from studies on fetal and adult bone marrow of normal and leukemia patients, it has been confirmed that FcγR is not expressed in non-committed progenitor CD34⁺ cells (Olweus J. et al. CD64/Fc Gamma RI is a granulo-monocytic lineage marker on CD34⁺hematopoietic progenitor cells. Blood. 85:2402-13, 1995; Aoki Y., et al. Identification of CD34⁺ and CD34⁻ leukemia-initiating cells in MLL-rearranged human acute lymphoblastic leukemia. Blood. 125:967-80, 2015). Accordingly, a negative selection of CD34⁺ stem cells that are untouched is achievable using the methods and reagents described herein. As is exemplified below, that result could be accomplished with just one mAbs, viz., biotinylated anti-CD3 mAb.

The principle of using clustered Fc on surfaces for binding of FcγRC and platelets can be used to advantage for another advantageous purpose. As disclosed, we have discovered that treatment of PBMC with RAM-FF can deplete all monocytes. Additionally, we have observed that platelets can be depleted with constructs with clustered Fc regions on their surfaces. Accordingly, PBMC and other similar mixtures can be treated with such agents to create PBMC preparations and mixures devoid of FcR bearing components. Human IgG bound to surfaces in a manner that brings their Fc fragments into close proximity would be ideal for binding to FcγR bearing components. The removal of such bearers of FcγR could readily be accomplished by passage of PBMC over densely bound human IgG adsorbents, with particles that are magnetic or buoyant and simply by labeling such entities so that their density is altered in a way that makes centrifugal methods of separating based on differential densities of cellular entities possible.

In summary, we have discovered that FcγR on FcγRC can be used advantageously for negative selections of naïve T cells using only a single antibody having specific reactivity to B cells and an Fc region that binds to FcγR. Two primary methods are disclosed that can each be undertaken in a direct labeling method, i.e., with a single key reagent bound to a solid support or in an indirect labeling method employing a single key reagent along with a surface or solid support, such as a common capture agent, that is capable of causing that key reagent to bind to it in a closely packed fashion. A variety of separation methods can be employed.

In addition to the straightforward binding of B cells, the mechanisms we believe are operative in the labeling of FcγRC are: (i) labeling of FcγRC with clustered Fc on solid supports when antibodies against B cells such as anti CD19 or CD20 are used; (ii) in the case of using anti-B cell surface Ig, e.g., mouse anti-human IgG, the fact that the mouse anti-human IgG also can bind to immunoglobulins bound to FcγRC and likely stabilize those binding reactions via crosslinking of near neighbor immunoglobulins and thus enhancing the binding constant of FcγR-antibody reactions; and (iii) if using anti-CD32, the antibody can bind to the FcγRII via the Fab paratope and to all FcγR via the Fc region (FIG. 1). As described above mechanism “2” is sensitive to free IgG in the system and, hence, is best undertaken in the lower levels of endogenous IgG found in unwashed PBMC samples. It appears that low affinity FcγR can also play a role in ‘labeling’ FcγRC when they interact with Fc regions that are in close proximity to each other via enhancement of their affinity constant. Thus, it is be possible, by altering levels of mAb combined with a PBMC, to selectively label just high affinity FcγR. That ability enables the isolation of naïve NK cells and B cells.

In addition to the above methods that in effect take advantage of the ‘specific binding pair reaction’ we have observed between FcR and clustered Fc on surfaces, we also disclose methods to immune-specifically target FcγR as a means of purifying either untouched or naïve T cells.

The presently claimed methods and compositions can be used to advantage in the preparation of cells for CAR T therapy. The invention also provides means for negative selection of CD34⁺ which also have significant therapeutic potential.

Finally, in view of the information provided herein, recombinant molecules could be engineered to perform the function that mAbs play via generation of one or more polypeptide sequences that mimic the FcγR binding region of those antibodies that bind to FcγR and a molecular entity (combined or independent) that can specifically bind a B cell or some other specificity in accordance with this disclosure.

The following materials and methods are provided to facilitate the practice of the present invention.
Magnetic particles-ferrofluid (FF): All FF was manufactured by BioMagnetic Solution (State College, PA). BSA/HSA-FF was synthesized by coating the FF with bovine serum albumin (BSA, Sigma) or human serum albumin (HSA, Akron Biotech, Boca Raton, Fla.), the size after coating is about 125-130 nm, which contain 84% of magnetic mass; RAM-FF was made by conjugating the rat-anti-mouse IgG1 mAb on BSA or HSA-FF using standard trout's reagent and sulfo-SMCC procedure (Thermo Fisher, Waltham, Mass.), and SA-FF was synthesized similarly by conjugating streptavidin (Agilent, Santa Clara, Calif.) to BSA or HSA-FF, the size is about 155-165 nm for both RAM- and SA-FF.
Antibodies and flow cytometry analysis: Mouse polyclonal antibody of anti-human IgG was obtained from Jackson Laboratory (Catalog # 209-005-082). Polyclonal goat anti-human IgG (Fab′)₂-biotin and Fab-biotin were purchased from Rockland Immunochemicals (Pottstown, Pa.), mAb to human CD3, CD19, IgG, CD20 and CD32 were from Absolute antibody (Boston, Mass.). Anti-human CD34 and CD56 antibody were from Biolegend (San Diego, Calif.), and the fluorescent antibody anti-CD45-FITC, CD3-PE, CD11b-PEcy5, CD19-PEcy5, CD34-PE and CD56-PE were from Biolegend. Cells before and after isolation were stained with fluorescent conjugated antibody, CD3, CD11b and CD19 positive cells were analyzed by gating on CD45 positive cells, isotype control was used to set up the analysis gates, and sample were analyzed using Guava® easyCyte™ Flow Cytometer (Luminex).
Cell and cell separation: Cells for many of the experiment were derived from apheresis product that was immediately centrifuged to remove plasma, washed with PBS containing 0.5% BSA by three cycles of centrifugation where the final pelleted cells were brought into suspension with CryoStor-CS10 (Biolife Solutions, Bothell, Wash.), aliquoted and frozen. CryoStor-CS10 is a serum free and protein free cell freezing medium. These aliquots are referred to as PBMC-Ig as they are substantially free of endogenous human IgG. Experiments also were performed using fresh apheresis products that were washed two times with either 1% BSA or HSA in PBS.
Direct negative cell selection procedure: A mouse polyclonal antibody of anti-human IgG (MAH-Ig) primarily of IgG₁ isotype was conjugated to BSA-FF using Trout's reagent and sulfo-SMCC following manufacturer's recommendation. The conjugation aimed on dense packing of antibodies on the surfaces of FF (4000-7000 mAb/particle). Similar conjugation was also performed with anti-human CD19 mAb. Frozen PBMC was diluted directly with equal volume of buffer (4% BSA in PBS) and incubated with 15 ug/mL (based on Fe) MAH-Ig-FF and cell concentrations of 2-10×10⁷ cell/mL for 20 min at room temperature. Since FF readily label cells by diffusion, no further mixing is needed during the 20-minute incubation. At the end of incubation, cells were further diluted to 2×10⁷/mL if needed and separated in a quadruple magnetic separator for 15 minutes. Cells that were not separated—negative fraction—were collected by aspiration. Analyses of negative fractions (untouched cells) were done by flow cytometry with Guava EasyCyte Plus Flow Cytometer using CD45, CD3 and CD11b antibodies. Purity and yield of the CD3⁺ T cells were analyzed.
Indirect negative selection procedure: Cells was first incubated with biotinylated antibody for 20 min, then SA-FF was added directly to cell mixture and incubated for another 15 min. Cells were then diluted to 2×10⁷/mL and subjected to magnetic separation.

The following examples are provided to illustrate certain embodiments of the invention. They are not intended to limit the invention in any way.

### EXAMPLE 1

### Isolation of Untouched CD3⁺ T Cells from Apheresis Products/PBMC using Direct Labeling Procedure with Anti-Human IgG Antibody

A mouse polyclonal antibody of anti-human IgG (mouse anti-human IgG (MAH-Ig) primarily of IgG₁ isotype was obtained from Jackson Laboratory (Catalog #209-005-082) and conjugated to FF using Trout's reagent and sulfo-SMCC. The level of antibody conjugation on MAH-Ig-FF was approximately 300 μg-500/μg of iron. For these 145 nm nanoparticles, this represents “dense packing” of antibodies on the particle surface, having approximately 4000-7000 mAb per particle.

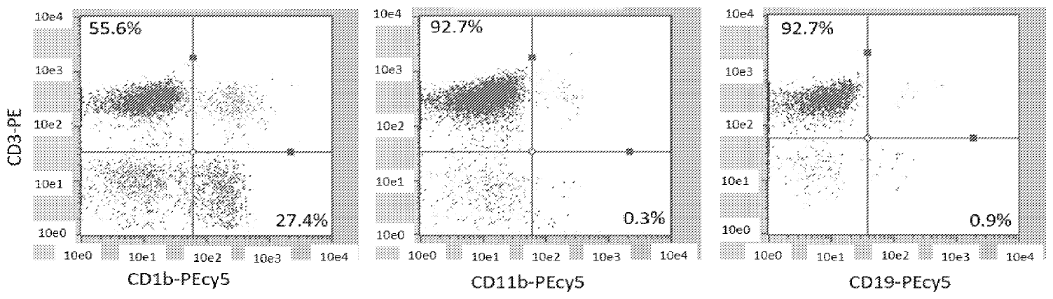

Thawed PBMC-Ig (endogenous Ig substantially removed) without further treatment were incubated with 15 μg/mL (based on iron concentration) MAH-Ig-FF and cell concentrations of 2-10×10⁷ cell/mL for 20 min at room temperature. Since FF readily label cells by diffusion, no further mixing is needed during the 20-minute incubation. At the end of incubation, cells were optionally diluted to 2×10⁷/mL, and separated in a quadrupole magnetic separator for 15 minutes. Cells that were not separated—negative fraction—were collected by aspiration. Analyses of negative fractions (untouched cells) was undertaken by flow cytometry using a Guava EasyCyte Plus Flow Cytometer, where T cells were identified by staining cells with anti-CD45-FITC and anti-CD3-PE. See FIG. 2. Purity of CD3⁺ T cells in multiple experiments was >95%.

From these results, it is apparent that a single antibody with binding specificity for human B cell surface Ig is sufficient to remove the majority of FcγRC as well as B cells. 95% purity of T cells is typically greater than those used for CAR T production in major clinical manufacturing organizations.

### EXAMPLE 2a

### Isolation of Untouched CD3⁺ T Cells from Apheresis Product/PBMC using Biotinylated Anti-Human IgG Antibody and SA-FF

Polyclonal mouse anti-human IgG (MAH-Ig) was biotinylated (MAH-Ig-Biotin) to a level of seven biotins/mAb, as measured by the HABA test. SA-FF (145 nm) was from BioMagnetic Solutions, State College, PA (Catalog #SAFF-109). PBMC-Ig at concentration of 2-10×10⁷ was incubated with a MAH-Ig-Biotin primarily of IgG₁ isotype at 2-4 μg/mL for 20 minutes at room temperature. SA-FF (15 μg/mL) was added to the cell mixture and incubated for another 15 minutes. At the end of incubation, cells were diluted to 2×10⁷/mL and then magnetically separated as above. As in example 1, cells in the negative fraction were analyzed by fluorescent staining and flow cytometry analysis and found in multiple experiments to be >95% untouched or naïve CD3⁺ T cells.

Given that the common capture agent, SA-FF, has no reaction with cells in PBMC-Ig and in particular with FcγR of any of those cells, t FcγRC are being removed in these experiments via one or more of the following mechanisms. Firstly, MAH-Ig-Biotin, when mixed with PBMC clearly labels B cells; it can also bind to high affinity FcγR and, if in sufficient concentrations, to lower affinity FcγR; MAH-Ig-Biotin cross links endogenous Igs that occupy FcγR, such cross linkingresulting in increased binding of those endogenous Igs to their FcγRs because of multivalent enhancement of affinity constants. Thus MAH-Ig-Biotin could be labeling FcγRC with biotin with high avidity. Another reaction that should be taking place when SA-FF is added to the system after the initial antibody incubation is that unbound MAH-Ig-Biotin will also simultaneously bind to SA-FF forming complexes that are capable of binding to FcγR. Hence, there are apparently multiple ways in which FcγRC are becoming magnetically labeled. Summarizing they are: (i) SA-FF binding to biotin on FcγRC via multivalent interactions which create very strong binding, i.e., avidity, (ii) SA-FF complexed with MAH-Ig-Biotin which interacts strongly with FcγR that are ‘empty’ or because of the likelihood of multivalent attachment, displace Ig in ‘filled’ FcγR, or (iii) MAH-Ig-Biotin binding to Ig occupying FcγR and cross linking the Ig via multivalent attachment generating sufficient binding energy such that cells are readily labeled by SA-FF.

### EXAMPLE 2b

### Isolation of Untouched CD3⁺ T Cells from Apheresis Product/PBMC using Biotinylated Anti-Human IgG (Fab′)₂ Fragment

Polyclonal biotinylated (Fab′)₂ fragment of goat anti-human IgG (FaHIg) was purchased from Rockland Immunochemicals (Pottstown, Pa.) and used in a similar experiment as described in Example 2a except a different amount of human IgG was added to cells before antibody labeling. The effect of PBMC-Ig pre-incubated with human IgG before addition of the biotinylated F(ab′)₂ fragment of anti-human IgG (FaHIg) is tabulated in Table 1. The B cell contamination (originally 15% in this preparation) increases significantly with increasing amounts of human IgG, which is to be expected as the labeling antibody is being neutralized by the added human IgG. On the other hand, CD11b⁺ cells (originally 29.3%) are significantly less affected. This suggests that complex binding reactions are at play in these experiments. For example, when there is no human IgG added, (Fab′)₂ most likely binds to Igs bound to FcγR and causing cross linking of those Igs and stabilizing their interaction with FcγR. Additionally, when human IgG is added in increasing amounts a second phenomenon is likely taking place, viz., (Fab′)₂ is binding to the IgG and forming complexes that strongly interact with FcγRC, thus leading to their being biotin labeled and subsequently removed either by the SA-FF or with SA-FF as part of the Ig-(Fab′)₂-SA-FF complexes. However, increasing amounts of IgG added would compete the interaction of (Fab′)₂ with B cell surface IgG and impact the depletion of B cells.

TABLE 1
Effect of adding human IgG prior to FαHIg on the
purity and yield of isolated naïve T cells

| [hIgG] at preincubation (μg/mL) | None | 125 | 250 | Before isolation |
|---|---|---|---|---|
| CD3⁺ cell purity, % | 95.3 | 88.7 | 84.3 | 54.94 |
| CD11b⁺ cells, % | 0.7 | 1.2 | 2.4 | 29.28 |
| B cell (CD19⁺), % | 0.9 | 8.3 | 13.4 | 15.15 |
| CD3⁺ cell yield, % | 57.0 | 73.0 | 78.9 | |

When experiments virtually identical to the above were undertaken with a biotinylated anti-CD19 mAb, high purity naïve T cells resulted. Pre-treatment with human IgG had little or no effect in those experiments, which confirmed our hypothesis regarding the mechanisms of interaction.

### EXAMPLE 3

### Isolation of Untouched CD3⁺ T Cells from Apheresis Product using Biotinylated Anti-Human CD32 and SA-FF

Because FcγR can be used to remove FcγRC as shown in the above examples, it should be possible to remove all such cells with antibodies to FcγR by a combination of antibody-epitope reactions and clustered Fc mediated FcRC magnetic labeling in the following way. Anti-CD32 antibody binds FcγRII which is widely expressed on leukocytes such as B cell/monocyte/granulocytes/platelets but not on T cells. This antibody could be used via an antibody—epitope reaction to label all FcγRII bearing cells including any B cells in the preparation. Binding will also occur between unbound biotinylated anti-CD32 antibody reacting with SA-FF, causing clustering of Fc on the FF which also binds avidly to FcγRC. This approach enables labeling of all gamma classes of FcR, in one case with anti-CD32 antibody and in the other case with the anti-CD32 antibody bound to a capture surface introduced into the system. After incubation in an appropriate separation apparatus, a high purity suspension of naïve T cells is obtained. To demonstrate the efficiency of this method, a biotinylated mouse mAb of IgG class having affinity for human CD32 was used. PBMC at concentrations of 2-10 x10⁷ were incubated with a biotinylated mouse anti-human CD32 mAb of IgG₁ isotype at 2 μg/mL for 15 minutes at room temperature—providing sufficient unbound mAb for subsequent reactions. SA-FF was added to the cell mixture and incubated for a further 15 minutes. Efficient labeling of FcRγC by either an antibody—epitope reaction or by clustered Fc on SA-FF was achieved. After separation of the labeled cells, the purity of untouched or naïve CD3⁺ T cells was >92% in the negative fraction.

### EXAMPLE 4

### Negative Selection of CD4⁺ or CD8⁺ T Cells from Apheresis Products or PBMC using Two Antibodies

Based on the previous examples, methods for preparation of naïve CD4⁺ or CD8⁺ cells were devised which included one additional mAb directed to either CD4 or CD8 depending on the T cell type to be negatively selected. Using the negative selection of CD4⁺ naïve cells to illustrate, two mAbs would be immobilized onto FF where one or both are reactive with FcγR, i.e., of the right subclass/isotype. Thus, if Example 1 were modified by having both anti-human IgG and anti-CD8 mAbs coupled to FF, such conjugates, in the absence of substantial levels of competing IgG, should deplete all of the FcγRC, B cell and CD8⁺ cells, leaving untouched CD4⁺ cells in the negative fraction. To obtain naïve CD8⁺ cells, the second mAb that is immobilized would be anti-CD4 rather than anti-CD8. Thus, the negative fraction would contain CD8⁺ cells.

In another approach, two biotinylated mAbs could be employed to accomplish the same results as described above, when a common capture agent such as SA-FF is employed. Thus producing naïve CD4⁺ cells would be accomplished by incubation of PBMC with biotinylated anti-CD8 and anti-B cell specific mAbs to label CD8⁺ cells, B cells and FcRC. Magnetic separation with SA-FF would result in negatively selected CD4⁺ cells. Based on the kinds of purities reported here, CD4⁺ cell purities in the mid to high 90%'s would be anticipated. This was confirmed experimentally using anti-CD19-biotin and anti-CD8 biotin in conjunction with SA-FF, CD4⁺ cells with purity of >92% were obtained.

### EXAMPLE 5

### Recovery of CD4⁺ and CD8⁺ Cells with two mAbs in a Single Magnetic Separation

Based on the clinical importance of CAR T constructs prepared by defined ratios of CD4⁺ and CD8⁺ cells, it would be desirable to accomplish such separations in an efficient manner. Using the procedure described here, the following scheme that utilizes only two mAb specificities would be reasonably expected to be capable of that task. One strategy for accomplishing such a separation would be to deplete all but CD4⁺ cells from PBMC-Ig, leaving CD4⁺ cells in the negative fraction and then to next retrieve CD8⁺ cells from the positively selected fraction by a simple extraction process.

For, example PBMC in the substantial absence of IgG can be incubated with an FcγR interacting biotinylated mAb, e.g., isotypes IgG_{1,} 2 or 3, specific for B cells and a desthiobiontinylated mAb of IgG subclass with non FcR binding antibody specific for CD8⁺ cells. Thus all FcγR bearing cells would be biotin labeled by the anti-B cell mAb and CD8⁺ cells would be labeled with desthiobiotinylated mAb. The addition of a common capture agent, e.g., SA-FF, after binding and separation, the CD8⁺ cells and all FcγRC would be positively separated and remaining negatively selected naïve CD4⁺ T cells readily retrieved. Based on the very significant difference in binding constants of biotin and desthiobiotin with SA (Hirsch J D, et al. Analytical Biochemistry. 308:343-357, 2002), it should be possible to release CD8⁺ cells from SA-FF by incubation with biotin that will displace the desthiobiotin mAb from their SA binding sites. In addition to desthiobiotin dissociation means via biotin displacement, methods for dissociating biotin—anti-biotin reactions are well known in the art (Lund, G. and Wegmann, T., U.S. Pat. No. 5,518,882; Brieden, J. and Dose, C. US Pat No. 20140113315A1). Hence, there are several ways in which to recover mAb labeled CD4⁺ or CD8⁺ cells in the above scheme.

For the recovery of CD8⁺ cells from SA-FF or other suitable dissociable binding pair, it would be advantageous to employ a magnetic separation device that separates magnetically labeled cells by spreading them out on a collection surface of sufficient area and magnetic gradient character such that they do not accumulate in a pile. If that was accomplished, as would be the case employing the magnetic separation system disclosed in patent publication WO 2016/183032 A1, CD8⁺ cells could be gently extracted while they are magnetically held on a collection surface. Alternatively, magnetically captured cells could be suspended with biotin (in the case of a SA-biotin system), release would occur. The mixture is then separated again, leaving CD8⁺ cells suspended that are easily recovered.

### EXAMPLE 6

### Isolation of Untouched B Cells from Apheresis Products by Indirect Labeling with Two mAbs

Based on results in the previous examples, we adapted these methods to successfully produce naïve B cells. In this approach, we take advantage of the fact that FcγRs expressed on leukocyte have different binding affinities. For example, monocytes, macrophages, DCs and granulocytes express high affinity FcγR (FcγRI) while B cells and NK cells express low affinity FcγR (FcγRIM for B cells, and FcγRIIC and FcγRIIIA for NK cells). While the high affinity FcγR is insensitive to valency, the low affinity FcγR prefers multimeric antibody in binding. Since B cells express a low affinity FcγR (FcγRIIB), reducing the concentration of mAbs used during the labeling step and adjusting the coating of SA on FF should promote high affinity reactions only, thereby preferentially labeling the high affinity FcγR bearing cells, such as monocytes, granulocytes and DCs in the reaction mixture via Fc binding on the mAbs used for targeting. Since CD3⁺ cells and NK cells also need to be targeted for removal, two mAbs suitable for this purpose, including but not limited to anti-CD3 and anti-CD16, could be employed. PBMC-Ig can be incubated with biotinylated anti-CD3 (at about 0.15 μg/mL) and anti-CD16 (at about 0.05 μg/mL) for 15 to 20 minutes to achieve appropriate labeling levels. All undesired cell types can then be removed with a common capture magnetic nanoparticle or some other suitable agent well known in the art. By these methods the recovery of untouched B cells in high purity is achieved.

### EXAMPLE 7

### Isolation of Untouched NK cells from Apheresis Products or PBMC using Two Antibodies

5-10% of PBMC are NK cells which form a major component of the revolutionary CAR-T immunotherapy which provides one of the most effective anti-cancer agents known to date (Shimasaki N., et al. Nature Reviews Drug Discovery, 2020, 19:200-218). Isolation of naïve NK cells for genetic engineering with cancer antigens comprises an important initial step of CAR-T. Based upon the same concept detailed in Example 6, by limiting mAb concentrations during the labeling reaction and SA density on FF particle, in the substantial absence of competing IgG, isolated untouched NK cells can be achieved. In one approach, a combination of biotinylated mouse anti-human B cell antibody (such as anti-CD19 mAb) and a biotinylated mouse anti-human CD3 antibody, at least one reagent being of the IgG₁ isotype are employed. The antibodies would be added at a concentration of approximately 0.2 μg/mL in total (0.15 μg/mL of anti-CD3 mAb and 0.05 μg/mL of anti-B (CD19) mAb). Under these antibody limiting conditions, only high affinity FcγR binding occurs. After incubation with mAbs at that the above approximate concentrations, SA-FF would be added, and separations performed as described in Example 2. With this reduced mAb concentration, T cells, B cells, as well as those high affinity FcγRC will be magnetically separated, leaving NK cells in the negative fraction.

### EXAMPLE 8

### Isolation of Untouched CD34⁺Hematopoietic Stem Cells by Negative Selection

In widely used current protocols, CD34⁺ stem cells are isolated via positive selection with anti-CD34 mAbs. It would be desirable to be able to process such cells for transplants or for genetic engineered therapies starting with untouched or naïve cells. Using current practices, a negative selection could be accomplished, but would require 7-9 mAbs to remove all but CD34⁺ cells. From an economic viewpoint this is not a viable approach. On the other hand, from studies on fetal and adult bone marrow of normal and leukemia patients, it was confirmed that the FcγR is not expressed in non-committed progenitor CD34⁺ cells (Olweus J. et al. CD64/Fc Gamma RI is a granulo-monocytic lineage marker on CD34⁺hematopoietic progenitor cells. Blood. 85:2402-13, 1995; Aoki Y., et al. Identification of CD34⁺ and CD34⁻ leukemia-initiating cells in MLL-rearranged human acute lymphoblastic leukemia. Blood. 125:967-80, 2015).

Accordingly, starting with an appropriate choice of mAbs to deplete all cells but CD34⁺ naïve cells in PBMC, the following strategy could be applied: a biotinylated mAb—anti-CD3 of the IgG isotype—would be incubated with PBMC for approximately 20 minutes, thereby labeling all T cells and FcγR expressing cells. Negative selection of non-labeled cells could be accomplished with SA on a solid support such as FF. The supernatant of such a magnetic separation would be the enriched CD34⁺ cell population, as T cells, B cells and other FcγRC (including platelets) are magnetically separated out of the solution. In certain embodiments, the donor of the PBMC has been treated with G-CSF to induce hematopoietic stem cells to migrate from the bone marrow into peripheral blood.

In this embodiment, it would be important to thoroughly label all cells to be removed by offering the system adequate quantities of the biotinylated mAb as well as adequate incubation time as approximately 98-99% of nucleated cells need to be removed. Employment of high affinity mAbs at concentrations of 2 μg per mL of cell suspension and for incubation periods of approximately 20 minutes should be adequate in cases where cell concentrations are approximately 1×10⁸/mL. In this situation, there are approximately 80,000 mAbs/cell which should be more than adequate for the labeling and subsequent removal of such cells.

With such a large percentage of cells being depleted there is the danger that desired CD34⁺ cells could be entrained. Hence, it would be advisable to perform separations of this type at total cell concentrations less than about 3×10⁷ total cells/mL and possibly as low as 5×10⁶ total cells. In our experience, we have found that addition of sucrose at 1% dramatically decreases entrainment. Additionally, in employing magnetic separation, it would be advisable to remove undesired cells over a large area separation to minimize entrapment. To achieve that, it would be advisable to use a separation system such as that disclosed by Liberti et al, WO 2016/183032 Al. Not only does that system minimize cells being collected in piles, but it also offers a method to remove entrained cells by a process called therein “meniscus scrubbing”—a gentle means for removing entrained cells.

While certain of the preferred embodiments of the present invention have been described and specifically exemplified above, it is not intended that the invention be limited to such embodiments. All patents, patent applications, and publications cited herein are expressly incorporated by reference in their entirety for all purposes. Various modifications may be made thereto without departing from the scope and spirit of the present invention, as set forth in the following claims.

What is claimed:

1. A method of isolating a target cell fraction from a peripheral blood mononuclear cell (PBMC) preparation comprising at least T and B cells, said preparation substantially lacking endogenous or added IgG, said target cell surfaces being substantially free of Fc receptor, the method comprising:
(a) introducing into said PBMC preparation, a single immunologically active capture agent which simultaneously binds to both Fc receptor-bearing cells and to epitopes on B cells which are present in said preparation, said capture agent being operably linked to a ferrofluid comprising magnetically responsive particles, thereby forming a magnetic cluster of Fc receptor bearing cells selected from B cells, monocytes, granulocytes, and platelets,;
(b) isolating said magnetic cluster of Fc receptor-bearing cells and B cells from said preparation in a magnetic separator; and
(c) recovering the target cell fraction in an essentially naïve condition.

2. A method of isolating a target cell fraction from a peripheral blood mononuclear cell (PBMC) preparation comprising at least T and B cells, said preparation substantially lacking endogenous or added IgG, said target cell surfaces being substantially free of Fc receptor, the method comprising:
(a) introducing into said PBMC preparation a single immunologically active capture agent which simultaneously binds to both Fc receptor-bearing cells and to epitopes on B cells which are present in said preparation, said capture agent being operably linked to a first member of a specific binding pair;
b) contacting the preparation of step a) with a ferrofluid comprising magnetically responsive particles operably linked to a second binding member, under conditions where a specific binding pair forms between said first and second binding pair members thereby forming a magnetic cluster of Fc receptor bearing cells selected from B cells, monocytes, granulocytes, and platelets;
c) isolating said magnetic cluster of Fc receptor-bearing cells and B cells from said preparation in a magnetic separator; and
(d) recovering the target cell fraction in an essentially naïve condition.

3. A method of isolating a target cell fraction from a peripheral blood mononuclear cell (PBMC) preparation comprising at least T and B cells substantially free of endogenous IgG, said target cell surfaces being substantially free of Fc receptor, the method comprising:
(a) introducing into said PBMC preparation an anti-human IgG and a capture agent comprising a first member of a specific binding pair and a Fab or F(ab)′₂ which binds to epitopes on B cells;
b) contacting the preparation of step a) with a ferrofluid comprising magnetically responsive particles operably linked to a second binding pair member, under conditions where a specific binding pair forms between said first and second binding pair members, thereby forming a magnetic cluster of Fc receptor bearing cells selected from B cells, monocytes, granulocytes, and platelets;
c) isolating said capture agent-bound Fc receptor-bearing cells and B cells from said preparation in a magnetic separator; and
(d) recovering the target cell fraction in an essentially naïve condition.

4. The method of any of claim 1, 2 or 3 wherein, the target cells are CD3⁺ T cells.

5. The method of any of one of claims 1 to 4, wherein said capture agent is an antibody or immunologically active fragment thereof having an Fab region which immunospecifically binds an epitope on B cells, and an Fc region which binds FcγR on non-target cells.

6. The method of claim 5, wherein FcγR bearing non-target cells are selected from monocytes, granulocytes, macrophages, dendritic cells, and NK cells.

7. The methods of claim 5, wherein the Fab region binds a B cell epitope selected from CD19, CD20, IgG and CD32.

8. The method of any one of claims 1 to 7, wherein the immunologically active capture agent is a monoclonal IgG antibody of mouse or human origin and comprises Fc regions which are bound by human FcγR.

9. The method of claim 8, wherein said monoclonal antibody is an IgG₁.

10. The method of any of the preceding claims, wherein said binding pair members are selected from biotin-streptavidin, receptor-ligand, agonist-antagonist, lectin-carbohydrate, avidin-biotin, biotin analog-avidin, desthiobiotin-streptavidin, desthiobiotin-avidin, iminobiotin-streptavidin, and iminobiotin-avidin.

11. The method of claim 1, wherein the capture agent comprises a ferrofluid having magnetically responsive particles are operably linked to a rat-anti-mouse IgG antibody or a mouse anti-human IgG antibody.

12. The method of claim 2, wherein said first or second member of said specific binding pair is biotin

13. The method of claim 12, wherein each antibody comprises between 3-7 biotin molecules.

14. The method of claim 2, wherein said first or second member of said specific binding pair is streptavidin.

15. A method of isolating naïve CD4⁺ T cells from a peripheral blood mononuclear cell (PBMC) preparation comprising at least T and B cells, said preparation substantially lacking endogenous or added IgG, said CD4⁺ T cell surfaces being substantially free of Fc receptor, the method comprising:
(a) introducing into said PBMC preparation a first immunologically active capture agent which simultaneously binds to both Fc receptor-bearing cells and to epitopes on B cells and a second immunologically active capture agent which binds to CD8⁺ T cells, each of said first and second immunologically active capture agents being operably linked to magnetically responsive particles present in ferrofluid;
(b) isolating said capture agent-bound Fc receptor-bearing cells, B cells and CD8+ cells from said preparation in a magnetic separator; and
(c) recovering the CD4⁺ T cells in an essentially naïve condition.

16. A method of isolating naïve CD8⁺ T cells from a peripheral blood mononuclear cell (PBMC) preparation, said preparation substantially lacking endogenous or added IgG, said CD8⁺ T cell surfaces being substantially free of Fc receptor, the method comprising:
(a) introducing into said PBMC preparation a first immunologically active capture agent which simultaneously binds to both Fc receptor-bearing cells and to epitopes on B cells and a second immunologically active capture agent which binds to CD4⁺ T cells, each of said first and second immunologically active capture agents being operably linked to magnetically responsive particles present in ferrofluid, said CD4⁺ T cells, said B cells and said Fc receptor bearing cells forming a magnetic cluster;
(b) isolating said magnetic cluster of cells from said preparation in a magnetic separator; and
(c) recovering the CD8⁺ T cells in an essentially naïve condition.

17. A method of isolating naïve NK cells from a peripheral blood mononuclear cell (PBMC) preparation, said preparation substantially lacking endogenous or added IgG, under conditions suitable for high affinity Fc-receptor binding, the method comprising:
(a) introducing into said PBMC preparation a first immunologically active capture agent which simultaneously binds to both Fc receptor-bearing cells and to epitopes on B cells and a second immunologically active capture agent which binds to CD3⁺ T cells under conditions suitable for high affinity FcR binding , each of said first and second immunologically active capture agents being operably linked to magnetically responsive particles present in ferrofluid and forming magnetic clusters of Fc-receptor bearing cells, B cells and CD3⁺ cells;
(b) isolating said magnetic cell cluster from said preparation in a magnetic separator; and
(c) recovering the NK cells in an essentially naïve condition.

18. A method of isolating naïve CD34⁺ stem cells from a peripheral blood mononuclear cell (PBMC) preparation comprising at least T and B cells, said preparation substantially lacking endogenous or added IgG, said CD34⁺ cell surfaces being substantially free of Fc receptor, the method comprising:
(a) introducing into said PBMC preparation an immunologically active capture agent which simultaneously binds to both Fc receptor-bearing cells and to epitopes on T cells, said immunologically active capture agent being operably linked to magnetically responsive particles present in ferrofluid;
(b) isolating said capture agent-bound Fc receptor-bearing cells, and T cells from said preparation in a magnetic separator; and
(c) recovering the CD34⁺ stem cells cells in an essentially naïve, untouched condition.

19. The method of claim 18, wherein said PBMC are isolated from a donor treated with G-CSF to cause hematopoietic stem cells to migrate from the bone marrow into peripheral blood.

20. The claim of 18, wherein the monoclonal antibody and streptavidin are present in concentrations which promote interactions with FcγRI.

21. The method of claim 1 or claim 2, wherein said ferrofluid comprises 4000-7000 monoclonal antibodies per particle.

22. The method of claim 17, where high affinity FcR binding conditions are promoted by reducing the concentration of monoclonal antibody to about 0.2 μg/ml.
